(19) United States
(12) Patent Application Publication
(10) Pub. No.: US 2015/0182668 A1
(43) Pub. Date: Jul. 2, 2015

(54) Electrospun PTFE Encapsulated Stent & Method of Manufacture

(71) Applicant: Zeus Industrial Products, Inc., Orangeburg, SC (US)

(72) Inventors: Robert L. Ballard, Orangeburg, SC (US); Bruce L. Anneaux, Lexington, SC (US); Sabrina D. Puckett, Lexington, SC (US); Joshua L. Manasco, West Columbia, SC (US); David P. Garner, Lexington, SC (US)

(21) Appl. No.: 14/631,909

(22) Filed: Feb. 26, 2015

Related U.S. Application Data
(63) Continuation of application No. 13/743,668, filed on Jan. 17, 2013.
Application No. 13/743,668 is a continuation-in-part of application No. 13/446,300, filed on Apr. 13, 2012.
Application No. 13/446,300 is a continuation of application No. 12/689,334, filed on Jan. 19, 2010, now Pat. No. 8,178,030.
Application No. 13/743,668 is a continuation-in-part of application No. 13/564,925, filed on Aug. 2, 2012.
Application No. 13/564,925 is a continuation of application No. 12/852,989, filed on Aug. 9, 2010, now Pat. No. 8,257,640.
Application No. 13/743,668 is a continuation-in-part of application No. 13/564,927, filed on Aug. 2, 2012, now Pat. No. 9,034,031.
Application No. 13/564,927 is a continuation of application No. 12/852,993, filed on Aug. 9, 2010, now Pat. No. 8,262,979.
Application No. 13/743,668 is a continuation-in-part of application No. 13/272,412, filed on Oct. 13, 2011, now Pat. No. 8,685,424.
Application No. 13/743,668 is a continuation-in-part of application No. 13/625,548, filed on Sep. 24, 2012.
(60) Provisional application No. 61/145,309, filed on Jan. 16, 2009.
(60) Provisional application No. 61/256,349, filed on Oct. 30, 2009.
(60) Provisional application No. 61/232,252, filed on Aug. 7, 2009.
(60) Provisional application No. 61/232,252, filed on Aug. 7, 2009.
(60) Provisional application No. 61/393,128, filed on Oct. 14, 2010.
(60) Provisional application No. 61/538,402, filed on Sep. 23, 2011.

(51) Int. Cl. A61L 31/04; A61F 2/07; A61L 31/14

(52) U.S. Cl. CPC A61L 31/041; A61L 31/146; A61F 2/07; A61F 2210/0071; A61F 2002/075

(57) ABSTRACT

A stent or other prosthesis may be formed by encapsulating a scaffold or frame with a polymer coating. The polymer coating may consist of layers of electrospun polytetrafluoroethylne (PTFE). Electrospun PTFE of certain porosities may permit endothelial cell growth within the prosthesis. The stent may be applicable to stents designed for the central venous system, peripheral vascular stents, abdominal aortic aneurism stents, bronchial stents, esophageal stents, biliary stents, or any other stent.

### CROSS-REFERENCE TO RELATED APPLICATIONS

This application is a continuation of U.S. patent application Ser. No. 13/743,668, filed Jan. 17, 2013;

Which application is a continuation-in-part of each of the following:

U.S. patent application Ser. No. 13/446,300, filed Apr. 13, 2012, which application is a continuation of U.S. patent application Ser. No. 12/689,334, filed Jan. 19, 2010, (now U.S. Pat. No. 8,178,030), which application claims the benefit of U.S. Provisional Patent Applications Nos. 61/145,309, filed Jan. 16, 2009, and 61/256,349, filed Oct. 30, 2009;

U.S. patent application Ser. No. 13/564,925, filed Aug. 2, 2012, which application is a continuation of U.S. patent application Ser. No. 12/852,989, filed Aug. 9, 2010, (now U.S. Pat. No. 8,257,640), which application claims the benefit of U.S. Provisional Patent Application No. 61/232,252, filed Aug. 7, 2009;

U.S. patent application Ser. No. 13/564,927, filed Aug. 2, 2012, which application is a continuation of U.S. patent application Ser. No. 12/852,993, filed Aug. 9, 2010, (now U.S. Pat. No. 8,262,979), which application claims the benefit of U.S. Provisional Patent Application No. 61/232,252, filed Aug. 7, 2009;

U.S. patent application Ser. No. 13/272,412, filed Oct. 13, 2011, which application claims the benefit of U.S. Provisional Patent Application No. 61/393,128, filed Oct. 14, 2010; and

U.S. patent application Ser. No. 13/625,548, filed Sep. 24, 2012, which application claims the benefit of U.S. Provisional Patent Application No. 61/538,402, filed Sep. 23, 2011.

### TECHNICAL FIELD

The present disclosure relates generally to medical devices. More specifically, the present disclosure relates to stents or other prostheses.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a front elevation view of one embodiment of a stent.

FIG. 2A is a perspective view of a covered stent.

FIG. 2B is a cross sectional view of the stent of FIG. 2A along the plane 2B-2B.

FIG. 3 illustrates one embodiment of a stent deployed in a body lumen.

FIGS. 4A-4B are scanning electron micrograph (SEM) images of embodiment of an electrospun PTFE outer covering for a stent.

FIGS. 5A-5B are SEM images of an electrospun PTFE inner layer of the covering of the stent of FIG. 4A-4B.

FIGS. 6A-6B are SEM images of an electrospun PTFE outer covering of another embodiment of a stent.

FIGS. 7A-7B are SEM images of an electrospun PTFE inner layer of the covering of the stent of FIG. 6A-6B.

### DETAILED DESCRIPTION

The entire disclosures of U.S. patent application Ser. No. 13/743,668, Filed Jan. 17, 2013, U.S. patent application Ser. No. 12/689,334, filed Jan. 19, 2010 (now U.S. Pat. No. 8,178,030), U.S. Provisional Patent Application Ser. No. 61/145,309, filed Jan. 16, 2009, U.S. Provisional Patent Application Ser. No. 61/256,349, filed Oct. 30, 2009, U.S. patent application Ser. No. 12/852,989, filed Aug. 9, 2010, (now U.S. Pat. No. 8,257,640), U.S. Provisional Patent Application No. 61/232,252, filed Aug. 7, 2009, U.S. patent application Ser. No. 12/852,993, filed Aug. 9, 2010, (now U.S. Pat. No. 8,262,979), U.S. patent application Ser. No. 13/272,412, filed Oct. 13, 2011, U.S. Provisional Patent Application Ser. No. 61/393,128, filed Oct. 14, 2010, U.S. patent application Ser. No. 13/625,548, filed Sep. 24, 2012, and U.S. Provisional Patent Application No. 61/538,402, filed Sep. 23, 2011, are incorporated herein by this reference as if set forth in their entireties.

Stents may be deployed in various body lumens for a variety of purposes. Stents may be deployed, for example, in the central venous system for a variety of therapeutic purposes including the treatment of occlusions within the lumens of that system. It will be appreciated that the current disclosure may be applicable to stents designed for the central venous (CV) system, peripheral vascular (PV abdominal aortic aneurism (AAA) stents, bronchial stents, esophageal stents, biliary stents, or any other stent. Further, the present disclosure may equally be applicable to other prosthesis such as grafts. Thus, the disclosure provided below in connection with specific examples of stents may apply analogously to other prostheses.

It will be readily understood that the components of the embodiments as generally described and illustrated in the Figures herein could be arranged and designed in a wide variety of different configurations. Thus, the following more detailed description of various embodiments, as represented in the Figures, is not intended to limit the scope of the disclosure, but is merely representative of various embodiments. While the various aspects of the embodiments are presented in drawings, the drawings are not necessarily drawn to scale unless specifically indicated.

The phrases connected to, coupled to, and in communication refer to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The directional terms proximal and distal are used herein to reference opposite locations on a stent. The proximal end of a stent is defined as the end of the stent closest to the practitioner when the stent is disposed within a deployment device which is being used by the practitioner. The distal end is the end opposite the proximal end, along the longitudinal direction of the stent, or the end furthest from the practitioner. It is understood that, as used in the art, these terms may have different meanings once the stent is deployed (i.e. the proximal end may refer to the end closest to the head or heart of the patient depending on application). For consistency, as used herein, the ends of the stent labeled proximal and distal prior to deployment remain the same regardless of whether the stent is deployed. The longitudinal direction of the stent is the direction along the axis of a generally tubular stent. In embodiments where a stent is composed of a metal structure coupled to a polymer layer, the metal structure is referred to as the scaffolding and the polymer layer as the coating. The term “coating” refers to a covering, typically fabric covering, that covers the scaffolding (or other frame) and, in many embodiments, encapsulates the frame inside and outside of the frame. “Coating” may refer to a single polymer, multiple layers of the same polymer, or layers comprising distinct polymers used in combination.

Lumens within the central venous system are generally lined with endothelial cells. This lining of endothelial cells throughout the central venous system makes up the endothelium. The endothelium acts as an interface between blood flowing through the lumens of the central venous system and the inner walls of the lumens. The endothelium, among other functions, reduces or prevents turbulent blood flow within the lumen.

A therapeutic stent which includes a coating of porous or semi-porous material may permit the formation of an endothelial layer on the inside surface of the stent. A stent which permits the formation of the endothelium within the stent may further promote healing at the therapeutic region. For example, a stent coated with endothelial cells may be more consistent with the surrounding body lumens, thereby resulting in less turbulent blood flow or a decreased risk of thrombosis, or the formation of blood clots. A stent which permits the formation of an endothelial layer on the inside surface of the stent may therefore be particularly biocompatible, resulting in less trauma at the point of application and fewer side effects.

Electrospun polytetrafluoroethylene (PTFE) may be used as a stent coating where endothelial cell growth is desirable. Electrospinning refers to a process for forming mats, tubes, or other shapes by depositing small strings of PTFE on collection surfaces using electric potential. The electrospinning process controls the thickness, density, porosity, and other characteristics of the PTFE so formed. Electrospinning of PTFE is described in U.S. Pat. No. 8,178,030, which is incorporated herein in its entirety by this reference.

The present disclosure relates to a stent which has, in certain embodiments, metal scaffolding coated with at least one layer of electrospun PTFE. It will be appreciated that, though particular structures and coatings are described below, any feature of the scaffolding or coating described below may be combined with any other disclosed feature without departing from the scope of the current disclosure.

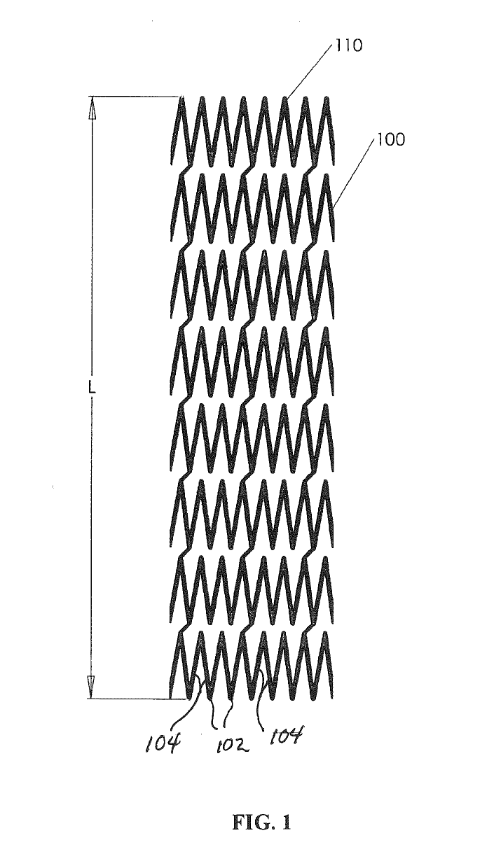

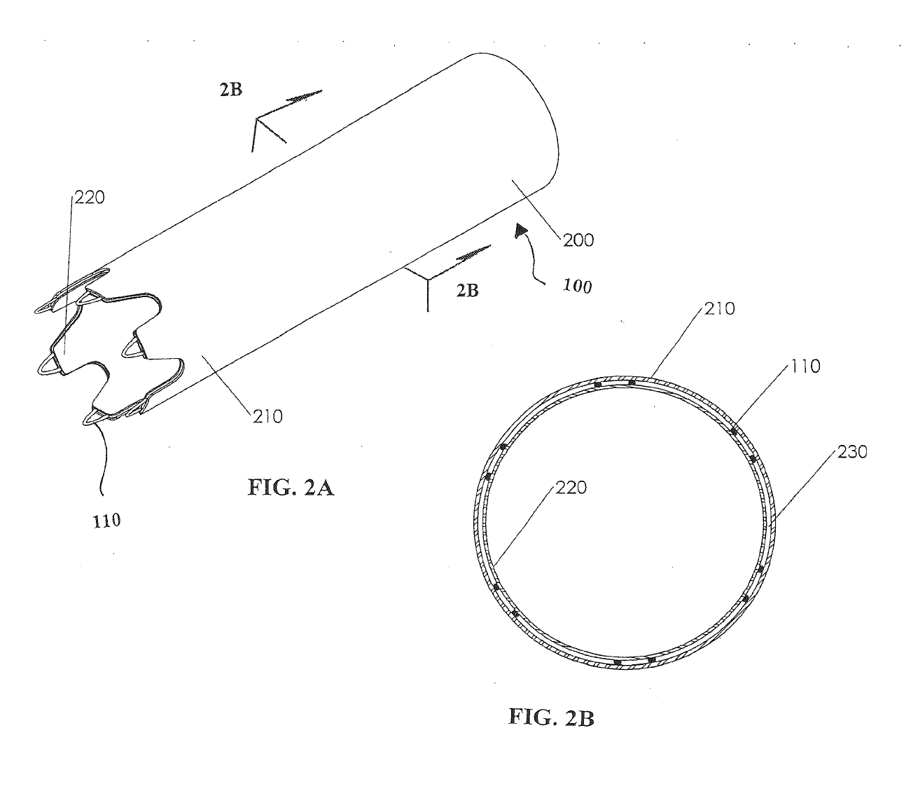

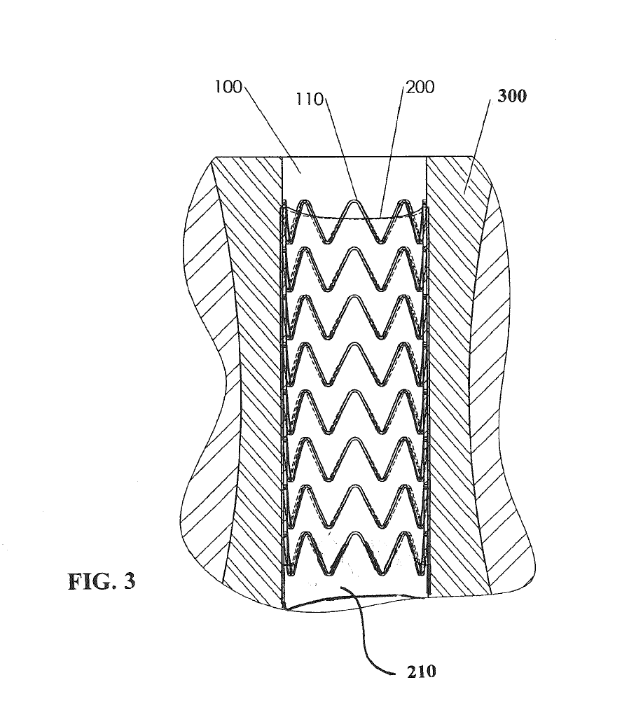

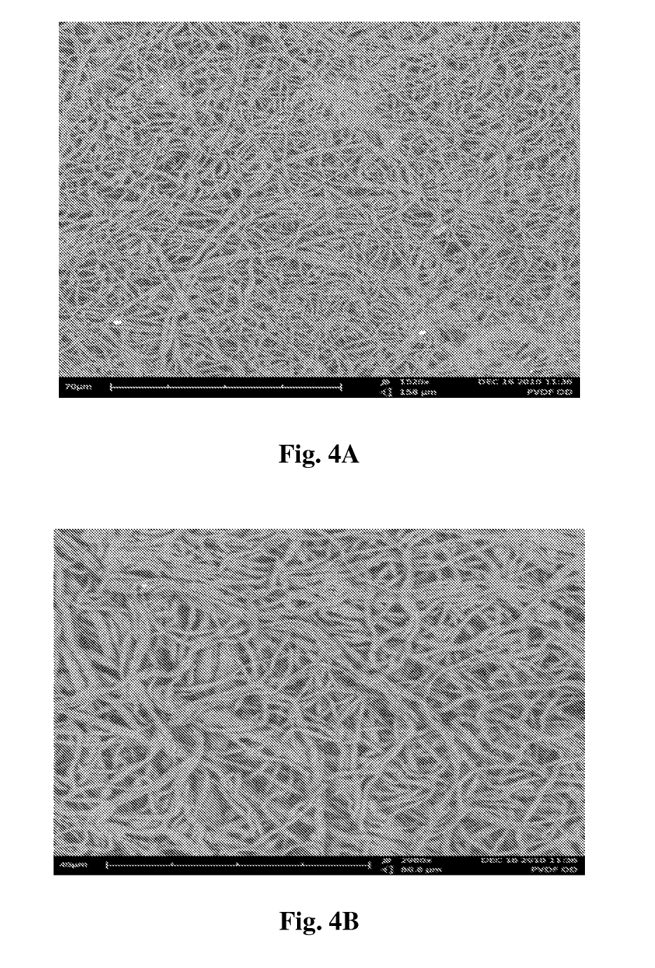

FIG. 1 shows a possible embodiment of a stent. FIGS. 2A and 2B illustrate an embodiment of a covered stent. FIG. 3 illustrates a stent deployed within a body lumen. Finally, FIGS. 4A-7B are scanning electron micrographs (SEMs) of possible coatings for stents. As indicated above, it will be understood that, regardless of whether the stent illustrated in any particular figure is illustrated with a particular coating, or any coating at all, any embodiment may be configured with any of the combinations of coatings shown or described herein.

FIG. 1 illustrates a front elevation view of an embodiment of a stent 100. The illustrated embodiment depicts one embodiment of a configuration for a metal wire 110 forming a scaffolding structure. This disclosure is not limited by any particular stent or frame or scaffolding structure.

Referring generally to FIG. 1, particular features of the illustrated stent are indicated. It will be appreciated that the numerals and designations used in any figure apply to analogous features in other illustrated embodiments, whether or not the feature is so identified in each figure. As generally shown in these Figures, the stent 100 may consist of a wire 110 shaped to form scaffolding. The wire 110 may be shaped in a wave-type configuration, the waves defining apexes 102 and arms 104 of the stent. The scaffolding may further be coupled to a covering layer (not pictured). Additionally, in some embodiments, any covering as disclosed herein may be applied to any type of scaffolding or stent frame, for example, laser cut stent frames, polymeric stent frames, wire scaffolding, and so forth.

The overall stent design may be configured to optimize desired radial force, crush profile, and strain profile. The stent design parameters may each be configured and tuned to create desired stent characteristics, as will be understood by those skilled in the art.

The stent 100 of FIG. 1 also has a length L. It will be appreciated that this length can vary depending on the desired application of the stent. In embodiments where the stent has flare zones at the ends, longer stents may or may not have proportionally longer flare zones. In some embodiments, this flare zone may be any length described above, regardless of the overall length of the stent.

It will be appreciated that the disclosed stent may be formed in a variety of sizes. In some embodiments, L may be from about 20 mm to about 200 mm. For example, in CV applications the stent may have a length, L, of from about 40 mm to 100 mm or any value between, for example, at least about 50 mm, 60 mm, 70 mm, 80 mm, or 90 mm. In PV applications the stent may have a length, L, of from about 25 mm to 150 mm or any value between, for example at least about 50 mm, 75 mm, 100 mm or 125 mm. The stent may also be longer or shorter than these exemplary values in other stent applications.

Likewise the stent may be formed with a variety of diameters. In some embodiments the midbody diameter of the stent may be from about 4 mm to about 40 mm. For example, in CV or PV applications the stent may have a midbody inside diameter of about 3 mm to 16 mm or any distance within this range such as between about 5 mm to 14 mm or between about 7 mm to about 10 mm.

The stent may or may not be configured with flared ends regardless of the midbody diameter employed. In some central venous embodiments the maximum diameter at the flared end will be between about 0.5 mm to about 2.5 mm greater than the midbody diameter. For example, the maximum diameter at the flared end may be between about 1 mm to about 2 mm, or alternatively between about 1.25 mm and about 1.5 mm, such as about 1.25 mm or about 1.5 mm greater than the midbody diameter.

Referring now to FIGS. 2A and 2B, in some embodiments the stent 100 may be comprised of a wire 110 which forms the scaffolding and a cover 200 coupled to the scaffolding. In some embodiments this cover may be comprised of a single layer, while in other embodiments it may be comprised of 2, 3, or more layers of material. One or more layers may be comprised of a polymer.

The illustrated embodiment has two cover layers, an outer layer 210 and an inner layer 220.

In some embodiments the outer layer 210, the inner layer 220, or both may be comprised of electrospun PTFE. Electrospun PTFE consists of tubes, mats, or other shapes of PTFE formed from randomly deposited strings of PTFE. As previously indicated, electrospinning of PTFE is described in U.S. Pat. No. 8,178,030 and other disclosure that have been incorporated herein, above. As described in the reference, electrospinning may comprise depositing a polymer on a collection surface, in the presence of an electrostatic field. In some instances the polymer may be electrostatically charged and may be discharged through one or more orifices.

Further information relative to electrospinning PTFE or other polymer is included below. The properties of electrospun PTFE, including density and porosity, may be controlled or influenced during the creation of the electrospun PTFE, through controlling the electrospinning process.

In some embodiments, a fiberizing agent is added to an aqueous dispersion of PTFE particles, to aid in the formation of PTFE fibers during the process of electrospinning the material. In some exemplary embodiments, polyethylene oxide (PEO) may be added as the fiberizing agent to the PTFE dispersion prior to electrospinning the material. In some instances the PEO may more readily dissolve in the PTFE dispersion if the PEO is first mixed with water. In some examples this increased solubility may reduce the time needed to dissolve PEO in aPTFE dispersion from as long as multiple days to as little as 30 minutes. In some embodiments, the PTFE dispersion may be discharged through an orifice to electrospin the PTFE. In an alternative embodiment, the PTFE dispersion is electrospun (e.g., into a fabric sheet or coating) using an open bath electrospinning apparatus. For example, the apparatus can comprise a wire, cylinder, spike, sharp edge, or similar geometry spinning electrode that creates a perturbation. For the open bath (trough) unit, the ejection volume is dependent upon the viscosity of the dispersion, the conductivity of the dispersion, the surface tension of the dispersion, the distance from bath to target, and the voltage. For either of the embodiments, after the material is electrospun onto a collector, the material may then be sintered as further described below. In some instances the sintering process will tend to set or harden the structure of the PTFE. Furthermore, sintering may also eliminate the water and PEO, resulting in a mat of substantially pure PTFE.

In one exemplary process, Poly(ethylene oxide) (300,000 kDa-40 grams) was added to 1 L aqueous dispersion of PTFE (˜60 wt % PTFE) with a 230 nm average particle size (for example, Daikin DX-9025, available from Daikin Industries, Ltd.) and allowed to gel (˜5 days). The material was then rolled to combine (˜10 rpm) for at least 48 hours to produce a viscous, off-white dispersion. The combined mixture was then allowed to sit or mix on a non-agitating jar roller until the solution achieved homogeneity. In other examples, the water, PEO, and PTFE amounts may be controlled to optimize the viscosity, PEO/PTFE ratio, or other properties of the mixture. In some instances adding water to the PEO before mixing with the PTFE dispersion may aid in reducing the number of large solid chunks in the mixture, lower the preparation time for the mixtures, and reduce the time needed for the combined mixture to solubilize.

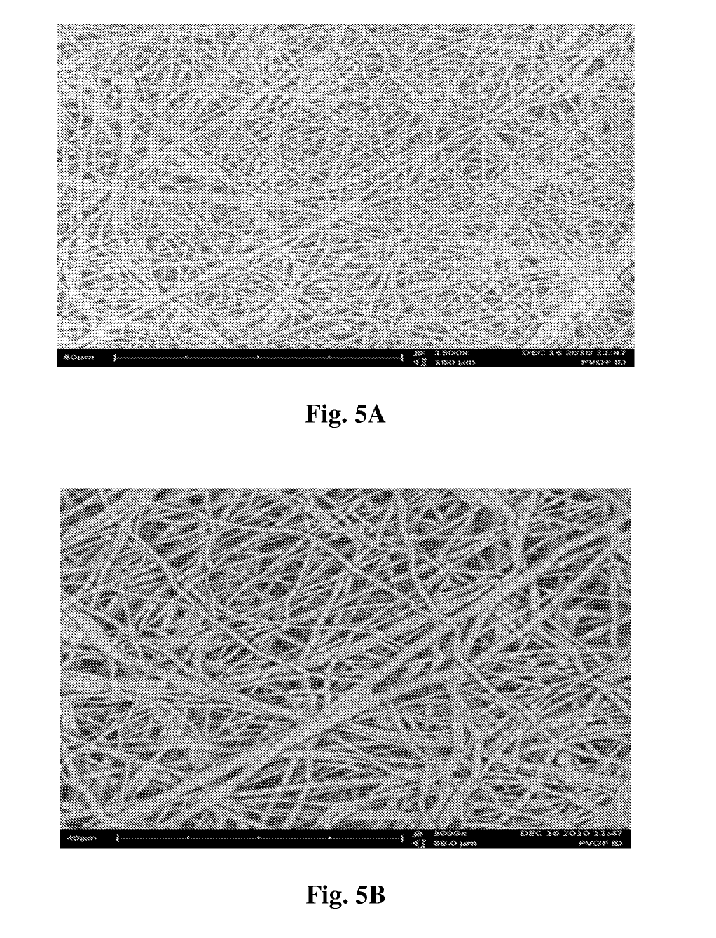

Nonwoven fabric composed of electrospun PTFE may have a microstructure composed of many fibers crossing each other at various and random points. The electrospinning process may control the thickness of this structure and, thereby the relative permeability of the fabric. As more and more strands of PTFE are electrospun onto a fabric, the fabric may both increase in thickness and decrease in permeability (due to successive layers of strands occluding the pores and openings of layers below). (This microstructure is shown in FIGS. 5A-7B, which are discussed in more detail below.)

The complex and random microstructure of electrospun PTFE presents a challenge to the direct measurement of the average pore size of the fabric. Average pore size can be indirectly determined by measuring the permeability of the fabric to fluids using known testing techniques and instruments. Once the permeability is determined, that measurement may be used to determine an effective pore size of the electrospun PTFE fabric. As used herein, the pore size of an electrospun PTFE fabric refers to the pore size of a fabric which corresponds to the permeability of the electrospun PTFE when measured using ASTM standard F316 for the permeability measurement. This standard is described in ASTM publication F316 Standard Test Methods for Pore Size Characteristics of Membrane Filters by Bubble Point and Mean Flow Pore Test, which is incorporated herein by reference.

In some applications it may be desirable to create a stent 100 with an outer layer 210 which is substantially impermeable. Such a layer may decrease the incidence of lumen tissue surrounding the stent growing into the stent. This may be desirable in applications where the stent is used to treat stenosis or other occlusions; an impermeable outer layer may prevent tissue from growing into the lumen of the stent and reblocking or restricting the body lumen. In some embodiments a substantially impermeable outer layer may be comprised of electrospun PTFE with an average pore size of about 0 microns to about 12 microns, more preferably between 0 and 5 microns, and most preferable less than 1 micron. In some embodiments, the impermeable layer may be a layer other than the outer layer, such as a tie layer, an intermediate layer or an inner layer. Furthermore, a substantially impermeable layer may be formed of fluorinated ethylene proplyene (FEP) which is applied, for example, as a film or dip coating. Furthermore, FEP may be electrospun with a small average pore size to create a substantially impermeable layer.

In other potential embodiments it may be desirable to create a stent with an outer layer 210 which is more porous. A porous outer layer 210 may permit healing and the integration of the prosthesis into the body. For instance, tissue of the surrounding lumen may grow into the porous outer diameter. This tissue ingrowth may permit healing at the therapy site. In some embodiments a porous outer layer 210 may be formed of electrospun PTFE.

In certain embodiments a relatively porous inner layer 220 may be desirable. This layer may or may not be used in conjunction with a substantially impermeable outer layer 210. A relatively porous inner layer may permit endothelial grown on the inside diameter of the stent 100 which may be desirable for healing, biocompatibility, and reducing turbulent blood flow within the stent. In some embodiments the inner layer may be comprised of electrospun PTFE with an average pore size of about 1 microns to about 12 microns, such as from about 2 microns to about 8 microns, or from about 3 microns to about 5 microns, or alternatively from about 3.5 to about 4.5 microns.

FIG. 2B illustrates a cross sectional view of a stent with an outer layer 210, an inner layer 220, and a wire scaffold 110. Additionally, the location between the outer layer 210 and the inner layer 220 is illustrated as 230. It will be appreciated that in embodiments where there are only two layers, there may not be a gap between the two layers, but the outer layer 210 and inner layer 220 may be in direct contact where they are not separated by the wire 110.

In other embodiments a third layer may be disposed in the location 230 between the outer layer 210 and the inner layer 220. In some embodiments this layer may be a tie layer configured to promote bonding between the outer layer 210 and the inner layer 220. In other embodiments the tie layer may further be configured to provide certain properties to the stent as a whole, such as stiffness or tensile strength. Furthermore, in embodiments where both the inner layer 220 and the outer layer 210 are porous in nature, the tie layer may be configured to create an impermeable layer between the two porous layers. In such embodiments the stent may permit cell growth and healing on both the inner and outer surfaces of the stent while still preventing tissue from outside the stent from growing into the lumen and occluding the lumen.

The tie layer may consist of any thermoplastic and may or may not be electrospun. In one embodiment, the tie layer may be expanded PTFE. In another it may be electrospun PTFE. In other embodiments it may be FEP, including electrospun FEP and FEP applied as a film or dip coating. Furthermore, the tie layer may consist of any of the following polymers or any other thermoplastic, such as polyamides, polyimides, epoxies, elastomers, silicones, polyurethanes, or the like, or other melt-processable fluoropolymers, including perfluoroalkoxy (PFA), fluorinated ethylene propylene (FEP), ethylene tetrafluoroethylene (ETFE), tetrafluoroethylene hexafluoropropylene vinylidene fluoride (THV)), polyvinylidene flouride (PVDF), or ethylene chlorotrifluoroethylene (ECTFE).

Regardless of the material, the tie layer may or may not be electrospun. Further, in certain embodiments the stent may consist of two or more tie layers. The tie layer may be formed in any manner known in the art and attached to the inner and outer layers in any manner known in the art. For example, the tie layer may comprise a sheet of material which is wrapped around the inner layer 220 or a tube of material which is slipped over the inner layer 220 which is then heat shrunk or otherwise bonded to the inner and outer layers. Further, in embodiments where the tie layer is electrospun, it may be electrospun directly onto the inner layer 220, the scaffolding, or both. In some instances the tie layer may be melted after the stent is constructed to bond the tie layer to adjacent layers of the stent covering.

Furthermore, tie layers may be configured to change the overall properties of the stent covering. For example, in some instances a cover comprised solely of electrospun PTFE (of the desired pore size) may not have desired tensile or burst strength. A tie layer comprised of a relatively stronger material may be used to reinforce the PTFE inner layer, the PTFE outer layer, or both. For example, in some instances FEP layers may be used to increase the material strength of the cover.

It will also be appreciated that one or more layers of electrospun PTFE may be used in connection with a scaffolding structure other than that disclosed herein. In other words, the disclosure above relating to covers, layers, tie layers, and related components is applicable to any type of scaffolding structure as well as to stents or grafts with no separate scaffolding structure at all.

FIG. 3 illustrates a cross section of a stent 100 disposed within a body lumen 300. The stent includes wire scaffolding 110 and a cover 200. In embodiments where the cover 200 is composed of an outer layer and an inner layer, the outer layer may be disposed adjacent to the body lumen while the inner layer may be disposed toward the inside portion of the body lumen. In particular, in embodiments where the stent is not substantially tubular in shape, the outer cover layer may be defined as the layer disposed adjacent the body lumen wall and the inner cover layer defined as the layer disposed toward the inner portion of the body lumen.

In some embodiments, a cover 200 may be formed by electrospinning a fabric onto a spinning mandrel. In other words, the collection device may comprise a mandrel, such as a substantially cylindrical mandrel, which rotates during the electrospinning process. Varying the speed at which the mandrel rotates may influence certain properties of the fabric. For example, in some embodiments, the density of the fabric (and thereby the average pore size) may be related to the rotational speed of the mandrel. Further, the directionality of the fibers, or the degree to which the fibers are deposited in a more controlled direction or manner, may be related to the rotational speed of the mandrel. In some instances a collection mandrel may rotate at rates between about 1 RPM and about 500 RPM during the elctrospinning process, including rates from about 1 RPM to about 50 RPM or at about 25 RPM. A fabric of electrospun PTFE formed onto a spinning mandrel may thus comprise a tubular fabric having no seam and substantially isotropic properties.

Once a fabric has been electrospun onto a mandrel, the fabric may then be sintered. In the case of PTFE, the fabric may be sintered at temperatures of about 385 degrees C., including temperatures from about 360 degrees C. to about 400 degrees C. Sintering may tend to set the structure of the PTFE, meaning individual particles of PTFE are melded into continuous fibers of PTFE. The melding of the PTFE at points of contact between fibers creates a three-dimensional structure of PTFE. Furthermore, sintering may evaporate any water or PEO mixed with the PTFE, resulting in a material comprised substantially of pure PTFE.

In some embodiments, a PTFE layer may be spun onto a mandrel and then sintered. Once the fabric is sintered, the tube of material may be removed from the mandrel, then slid back on the mandrel (to initially break any adherence of the fabric to the mandrel). In other instances, low friction coatings may alternatively or additionally be applied to the mandrel before the fabric is electrospun. Once the fabric is reapplied to the mandrel, a scaffolding can be placed over the mandrel and the fabric. A second layer of material may then be spun onto the scaffolding and the fabric, and subsequently sintered. Additionally layers may also be added.

In some instances, the layers may comprise a first layer of PTFE, a second layer of FEP, and a third layer of PTFE. The properties of each of these layers, including average pore size, may be controlled to form coating that inhibit growth of tissue through a particular layer or that permits endothelial growth on a particular layer.

In another example, a first layer of PTFE may be spun on a mandrel, sintered, removed from the mandrel, replaced and the mandrel, and a scaffolding structure applied. An FEP layer may then be applied by dipping, spraying, application of a film layer, electrospinning, or other processing. The FEP layer may or may not be sintered before applying an outer PTFE layer.

In another particular example, a first layer of PTFE may again be spun on a mandrel, sintered, removed, replaced, and a scaffolding structure applied. An FEP layer may then be applied as a film layer. An outer tube of PTFE (which may be formed separately by electrospinning onto a mandrel and sintering) may then be disposed over the FEP film layer. The entire construct may then be pressured, for example by applying a compression wrap. In some embodiments this wrap may comprise any suitable material, including a PTFE based material. In other embodiments a non-stick barrier, ie aluminum foil, may be wrapped around the construct before the compression wrap, to prevent the construct from adhering to the compression wrap.

The compressed layers may then be heated above the melting temperature of the FEP, but below the sintering temperature of the PTFE. For example, the melt temperature of the FEP may be from about 300 degrees C. to about 330 degrees C., including about 325 degrees C. PTFE may be sintered at temperatures from about 360 degrees C. to about 400 degrees C. Thus, the entire construct may be heated to an appropriate temperature such as about 325 degrees C. In some embodiments the construct may be held at this temperature for about 5 to about 10 minutes. This may allow the FEP to flow into the porous PTFE nanofiber layers surrounding the FEP. The joining of the FEP tie layer to the PTFE outer and inner cover layers may increase the strength of the finished covering. The construct may then be cooled and the compression wrap and the non-stick barrier discarded. The construct may then be removed from the mandrel.

A stent formed by the exemplary process described above may be configured with desired characteristics of porosity and strength. In some instances the FEP material may coat the PTFE nanofibers, but still allow for porosity which permits endothelial growth. The degree to which the FEP coats the PTFE may be controlled by the temperature and time of processing. The lower the temperature and/or the shorter the time the construct is held at temperature, the less the FEP may flow. In some instances a tie layer of FEP which is impervious the tissue growth through the layer may be formed by heating the construction only to about 260 degrees C.

Additionally, in some embodiments a stent may also include an extension cuff 210 (see FIG. 3) at one or both ends of the stent. The extension cuff 210 is just the coating material with no scaffold in between the inner and outer layer. The extension cuff may be present to provide easier attachment to a vessel in the body.

FIGS. 4A-5B are scanning electron micrograph (SEM) images of an exemplary embodiment of a stent covering. FIGS. 4A-4B are images of the outer layer of the covering while FIGS. 5A-5B are images of the inner layer of the covering. For each SEM, the electrospun PTFE was covered with a very thin layer of gold in order to make the structure visible on an SEM image.

FIG. 4A is an SEM image of the outer covering at 1500× (actually, 1520×) magnification, and FIG. 4B an SEM image at 3000× (actually, 2980×) magnification. Similarly, FIG. 5A is an image of the inner covering at 1500× magnification, FIG. 5B at 3000× magnification.

These SEM images reflect the microstructure of electrospun PTFE, depicting the randomly deposited criss-crossing fibers of PTFE that form the covering.

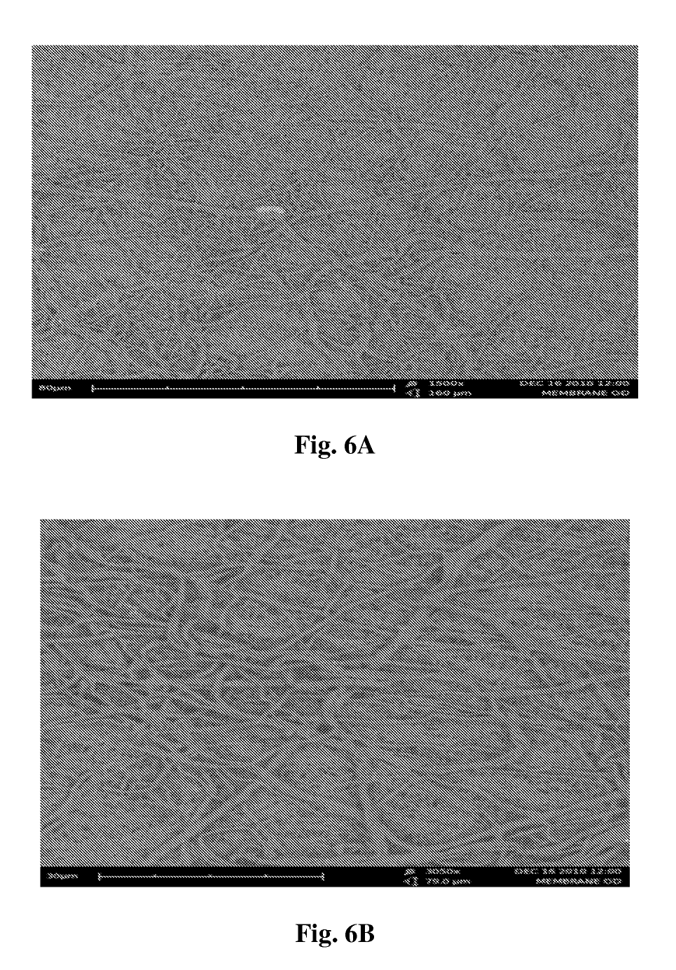

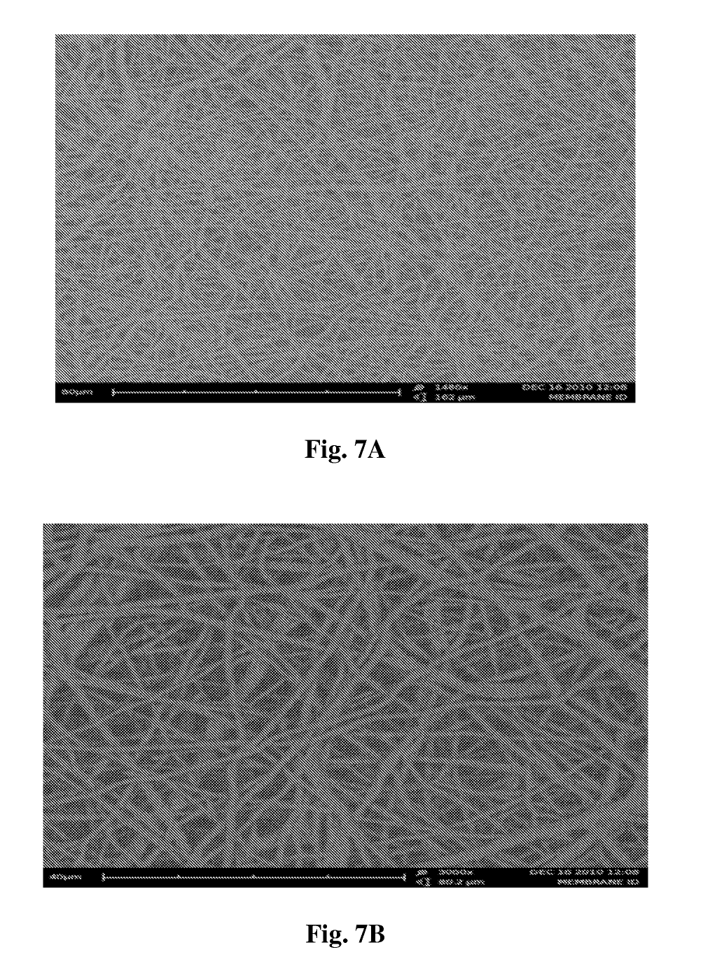

FIGS. 6A-7B are scanning electron micrograph (SEM) images of a second exemplary embodiment of a stent covering. FIGS. 6A-6B are images of the outer layer of the covering while FIGS. 7A-7B are images of the inner layer of the covering. Again, for each SEM, the electrospun PTFE was covered with a very thin layer of gold in order to make the structure visible on an SEM image.

FIG. 6A is an SEM image of the outer covering at 1500× magnification, and FIG. 6B an SEM image at 3000× (actually, 3050×) magnification. Similarly, FIG. 7A is an image of the inner covering at 1500× (actually, 1480×) magnification, and FIG. 7B at 3000× magnification.

While specific embodiments of stents have been illustrated and described, it is to be understood that the disclosure provided is not limited to the precise configuration and components disclosed. Various modifications, changes, and variations apparent to those of skill in the art having the benefit of this disclosure may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill, having the benefit of this disclosure, in the art that changes may be made to the details of the above-described embodiments without departing from the underlying principles of the disclosure herein

1-23. (canceled)

24. A vascular prosthesis, comprising: a tubular body comprising a first layer of electrospun polytetrafluoroethylene (PTFE); an impermeable layer, configured to be impermeable to tissue ingrowth through the impermeable layer; and a second layer of electrospun PTFE.

25. The vascular prosthesis of claim 24, wherein the impermeable layer comprises electrospun PTFE.

26. The vascular prosthesis of claim 24, wherein the impermeable layer is disposed between the first and second layers of electrospun PTFE.

27. The vascular prosthesis of claim 26, wherein the impermeable layer is in direct contact with at least one of the first and second layers of electrospun PTFE.

28. The vascular prosthesis of claim 24, wherein the impermeable layer comprises a tie layer disposed between at least two layers.

29. The vascular prosthesis of claim 28, wherein: the first layer of electrospun PTFE defines an outermost surface of the tubular body; the second layer of electrospun PTFE defines an innermost surface of the tubular body; and the impermeable layer is disposed between the first and second layers of electrospun PTFE.

30. The vascular prosthesis of claim 29, wherein the impermeable layer comprises a thermoplastic material.

31. The vascular prosthesis of claim 29, wherein the impermeable layer comprises fluorinated ethylene propylene (FEP).

32. The vascular prosthesis of claim 31, wherein the FEP is bonded to the first and second layers of electrospun PTFE by heating and compression.

33. The vascular prosthesis of claim 29, wherein at least one of the first and second layers of electrospun PTFE has an average pore size configured to permit cellular growth into the layer of electrospun PFTE.

34. The vascular prosthesis of claim 33, wherein at least one of the first and second layers of electrospun PTFE has an average pore size between about 2 microns and about 8 microns.

35. The vascular prosthesis of claim 29, wherein the material comprising the impermeable layer has greater tensile strength than at least one of the first and second layers of electrospun PTFE.

36. The vascular prosthesis of claim 24, wherein all the PTFE in the vascular prosthesis comprises deposited PTFE.

37. The vascular prosthesis of claim 36, wherein all the PTFE in the vascular prosthesis comprises electrospun PTFE fibers.

38. The vascular prosthesis of claim 24, further comprising a scaffolding structure coupled to the tubular body.

39. The vascular prosthesis of claim 38, wherein the scaffolding structure is disposed between adjacent layers of the tubular body.

40. A vascular prosthesis, comprising: a tubular body comprising two or more layers of material, wherein at least one layer of the tubular body comprises electrospun polytetrafluoroethylene (PTFE); and wherein all the PTFE in the vascular prosthesis comprises electrospun PTFE.

41. The vascular prosthesis of claim 40, wherein at least one layer of the tubular body is impermeable to tissue ingrowth through the impermeable layer.

42. The vascular prosthesis of claim 40, wherein at least one layer of electrospun PTFE has an average pore size configured to permit cellular growth into the layer of electrospun PTFE.

43. The vascular prosthesis of claim 40, wherein at least one layer of electrospun PTFE has an average pore size between about 2 microns and about 8 microns.

44. The vascular prosthesis of claim 40, further comprising a scaffolding structure coupled to the tubular body.

45. The vascular prosthesis of claim 40, wherein the tubular body comprises: a first layer of electrospun PTFE; a second layer of electrospun PTFE; and an impermeable layer disposed between the first and second layers of electrospun PTFE.

46. A medical appliance, comprising: a first layer comprising deposited polytetrafluoroethylene (PTFE), wherein all the PTFE in the medical appliance comprises deposited PTFE.

47. The medical appliance of claim 46, wherein at least a portion of the first layer comprises spun PTFE.

48. The medical appliance of claim 46, further comprising a second layer wherein the first layer comprises a first surface of the medical appliance and the second layer is coupled to the first layer.

49. The medical appliance of claim 48, further comprising a tie layer disposed between the first layer and the second layer.

50. The medical appliance of claim 49, wherein the tie layer is impermeable to tissue growth through the tie layer.
